(19) United States
(12) Patent Application Publication
(10) Pub. No.: US 2024/0065192 A1
(43) Pub. Date: Feb. 29, 2024

(54) METHOD TO ENRICH DESIRED TYPES IN SEED-DERIVED CROPS

(71) Applicant: Wisconsin Alumni Research Foundation, Madison, WI (US)

(72) Inventors: Shawn Kaeppler, Oregon, WI (US); Michael Petersen, Merrimac, WI (US)

(21) Appl. No.: 18/451,658

(22) Filed: Aug. 17, 2023

Related U.S. Application Data
(60) Provisional application No. 63/398,726, filed on Aug. 17, 2022.

(51) Int. Cl. A01H 1/04; A01H 6/28; C12N 15/82

(52) U.S. Cl. CPC A01H 1/045; A01H 6/28; C12N 15/8209

(57) ABSTRACT

The present invention provides cannabis plants, seeds, and pollen grains comprising a Y chromosome that comprises a marker gene. Methods of sorting cannabis seeds and pollen based on the presence of this marker gene are also provided.

### CROSS-REFERENCE TO RELATED APPLICATIONS

This application claims priority to U.S. Provisional Application No. 63/398,726 filed on Aug. 17, 2022, the contents of which are incorporated by reference in their entireties.

### SEQUENCE LISTING

This application includes a sequence listing in XML format titled “2023-08-17_960296.04427_WIPO_Sequence_listing_XML.xml”, which is 21,830 bytes in size and was created on Aug. 17, 2023. The sequence listing is electronically submitted with this application via Patent Center and is incorporated herein by reference in its entirety.

### BACKGROUND

The cannabis industry is one of the country's fastest-growing industries. Female cannabis plants produce flowers, which are the source of the lucrative cannabinoids tetrahydrocannabinol (THC) and cannabidiol (CBD), among many others. Male cannabis plants make pollen, which can be used to fertilize the female cannabis plants. However, when the female plants get fertilized, they use all their energy to produce seeds instead of making flowers, reducing their profitability.

Accordingly, cannabis growers commonly attempt to sex their plants as early as they can, destroying all or most of the male plants to maximize flower production. Sexing can be accomplished using morphological differences that become apparent as the plants mature or by performing DNA sequencing or a polymerase chain reaction (PCR)-based assay on a sample of plant tissue. While DNA sequencing and PCR can be performed on very young seedlings, these methods are expensive and time consuming, and are therefore only practical when applied to small numbers of plants. Alternatively, some growers use hormone treatments to produce male flowers on genetically female plants (masculinization) or to produce female flowers on genetically male plants (feminization). However, these methods are inefficient, difficult to scale, and can only produce progeny of a single sex. Thus, there is a growing need in the art for a higher throughput means of sexing cannabis.

### SUMMARY

In a first aspect, the present invention provides cannabis plants, seeds, and pollen grains comprising a Y chromosome that comprises a promoter operably linked to a marker gene.

In a second aspect, the present invention provides methods of sorting cannabis seeds or pollen. These methods allow one to control the sex of the plant or the relative proportions of male and female plants produced from the sorted seeds or pollen.

In a first embodiment, the methods are for sorting cannabis seeds. These methods comprise: (a) obtaining a plurality of cannabis seeds comprising seeds that comprise a Y chromosome comprising a marker gene; and (b) sorting the seeds to separate the seeds that comprise the Y chromosome comprising the marker gene from seeds that do not comprise the Y chromosome comprising the marker gene. In these methods, the marker gene must be expressed in seeds to allow for seed sorting.

In a second embodiment, the methods are for sorting cannabis pollen. These methods comprise: (a) obtaining a plurality of cannabis pollen grains comprising pollen grains that comprise a Y chromosome comprising a marker gene; and (b) sorting the pollen grains to separate the pollen grains that comprise the Y chromosome comprising the marker gene from the pollen grains that do not comprise the Y chromosome comprising the marker gene. In these methods, the marker gene must be expressed in pollen to allow for pollen sorting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows photographs of mature crushed (FIG. 1A) and intact (FIG. 1B) cannabis seeds from transformation experiments outlined in Example 1. The seed(s) on the left in each photograph depict positive expression of the RUBY (SEQ ID NO: 1) marker, and the seed(s) on the right in each photograph depict control seeds that are negative for expression of the RUBY (SEQ ID NO: 1) marker.

FIG. 2 shows a diagram of Plasmid RC6233A as outlined in Example 1.

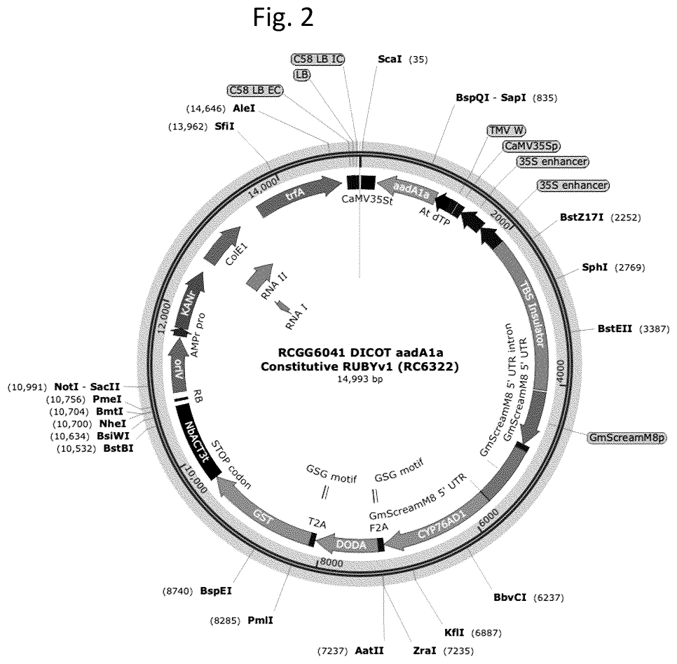

### DETAILED DESCRIPTION

The present invention provides cannabis plants, seeds, and pollen grains comprising a Y chromosome including a marker gene. Methods of sorting cannabis seeds and pollen based on the presence of this marker gene are also provided.

Cannabis is generally a dioecious plant in which the inheritance of sex chromosomes dictates the sex of the plant. Namely, cannabis plants that inherit two X chromosomes are female and cannabis plants that inherit one X chromosome and one Y chromosome are male. Thus, Y chromosomes are found only in male cannabis plants. Accordingly, the present inventors have designed methods for sexing cannabis seeds or pollen using a marker gene located on the Y chromosome. Their methods allow one to sort seeds/pollen into male and female pools, and then select an appropriate number of seeds/pollen grains from each of those pools to achieve the desired male to female ratio for a given application.

The sexing methods of the present invention offer several advantages over the methods of the prior art. For example, these methods are inexpensive, efficient, and can be used to produce populations of cannabis plants having the desired male to female ratio. Importantly, sexing cannabis at the seed/pollen stage rather than at the seedling stage prevents growers from wasting resources growing and culling excess male plants.

### Cannabis Plants, Seeds, and Pollen Grains:

In a first aspect, the present invention provides cannabis plants, seeds, and pollen grains comprising a Y chromosome that comprises a promoter operably linked to a marker gene.

As used herein, the term “cannabis” refers to a genus of flowering plants in the family Cannabaceae, which includes the species Cannabis sativa and Cannabis indica. These plants are also known as “hemp,” but this term is often used to refer only to varieties of cannabis that are cultivated for non-drug use. The terms “cannabis” and “hemp” are used interchangeably herein. The cannabis plant is used to produce cannabis fiber, cannabis seeds and their oils, cannabis leaves for use as vegetables and juice, and flowers (also known as buds), which are dried and used for medicinal and recreational purposes.

The term “plant” is used broadly herein to refer to a plant at any stage of development or the part of a plant, including a plant cutting, a plant cell, a plant cell culture, a plant organ, a plant tissue, or a plantlet. Particularly useful parts of a plant include harvestable parts (e.g., flowers) and parts that can be used for propagation of progeny plants (e.g., cuttings, seeds, pollen). A “seed” is a mature fertilized plant ovule, consisting of an embryo and its food store surrounded by a protective seed coat. “Pollen” is a powdery substance produced by male or masculinized female plants. Pollen consists of “pollen grains”, i.e., highly reduced microgametophytes, which produce male gametes. Pollen grains have a hard coat made of sporopollenin that protects the gametophytes as they move from the stamens to the pistil of flowering plants.

The cannabis plants, seeds, and pollen grains of the present invention comprise a marker gene on the Y chromosome, which allows them to be sexed based on expression of the marker protein encoded by the marker gene. As used herein, the term “marker gene” refers to a gene that encodes a marker protein that has a detectable phenotype in seeds and/or pollen. The marker gene used with the present invention may be natively found in cannabis and may even be found on the cannabis Y chromosome or may be artificially introduced (e.g., via random or targeted insertion). The term “native marker gene” or “native marker” refers to a gene native to cannabis that encodes a marker protein that has a detectable phenotype. In some embodiments, the native marker gene is a regulatory element that affects gene expression, such as a transcription factor or enhancer. The regulatory element may regulate expression of a gene that, when increased or decreased, results in a detectable phenotype. Thus, changes in expression of the regulatory element may have pleiotropic effects, leading to changes in expression of additional downstream genes. The detectable phenotype may result from changes in expression of the regulatory element and/or changes in expression of the downstream gene(s). In other embodiments, the native marker gene is not a regulatory element. Examples of native marker genes are WRINKLED (WRI1); native marker genes in the flavonoid biosynthesis pathway (e.g. KNAT7, NAC2, TTG2, and STK); and regulatory genes/transcription factors in the flavonoid biosynthesis pathway (e.g. C2H2-like, bZIP44, SHP1, GBF6, PPO, CHS, P, Z, and Bip).

In some embodiments, the artificially introduced marker gene or the artificially introduced native marker gene is inserted on the Y chromosome. In some embodiments, the artificially introduced marker gene or artificially introduced native marker gene is inserted on a chromosome other than the Y chromosome and is transposed onto the Y chromosome by transposable elements, such as Activator-Dissociation (Ac-Ds) transposable elements.

In embodiments where a native marker gene is artificially introduced, it may be necessary to significantly reduce or knock out the function of the endogenous native marker gene inherent to the plant because its expression may interfere with or confound the expression of, or the detection of the expression of, the artificially introduced native marker gene. In other embodiments, it may be desirable to simply reduce or knock out the function of the endogenous native marker gene even if no native marker gene is artificially introduced using genetic engineering techniques. Examples of methods to significantly reduce or knock out gene function are via methods known in the art, such as injection, particle bombardment, nanoparticles, encapsulation, or electroporation, or delivered via another cell or a virus that is then fused with the cell. In some embodiments, genetic engineering involves altering the nuclear genome of the cell. When new genetic material is introduced to the nuclear genome, it can be inserted randomly or targeted to a specific location (e.g., via homologous recombination). Suitable gene-editing reagents include, without limitation, engineered nucleases, such as meganucleases, zinc finger nucleases (ZFN), transcription activator-like effector nucleases (TALENs), and nucleic-acid guided nucleases (e.g., Cas9); guide nucleic acids; template nucleic acids; and reagents that facilitate the delivery of nucleases and nucleic acids to a cell (e.g., recombinant viruses, nanoparticles) may also be included. If there is more than a single copy of the endogenous native marker gene, it may be necessary to knock out or significantly reduce (knock down) function of all gene copies. Those of skill in the art are able to develop plants with particular genes knocked out as noted above. Those of skill in the art are also able to design methods of knocking down expression of a protein, by inhibitory RNA techniques including RNAi, shRNA, microRNA and the like.

Examples of detectable phenotypes include morphological differences (e.g., differences in length, width, volume, surface area, shape, surface texture, weight, viability) and detectable signals (e.g., color, luminescent signals, fluorescent signals, hyperspectral signals). In some embodiments, the marker gene encodes a fluorescent protein (e.g., RFP, GFP, YFP, CFP), chromogenic protein (e.g., meffRed, eforRed, aasPink, spisPink, scOrange), carotenoid protein (e.g., crtB, crtI, crtE), anthocyanin protein (e.g., PAL, CHS, F3H), or betalain protein (CYP76AD1, DODA, GT). The detectable phenotype of the marker protein should be detectable in seeds and/or pollen produced by the plant carrying the marker gene.

In U.S. Patent Publication No. 2021/0254083, which is hereby incorporated by reference in its entirety, the present inventors demonstrate that tdTomato and betalains can be expressed in cannabis and provide methods of generating gene edited or transgenic cannabis plants. These methods can be used to generate and select for transgenic or gene edited cannabis plants for use with the present invention. TdTomato is an exceptionally bright red fluorescent protein with an emission wavelength of 581 nm. Betalains are red and yellow water-soluble plant pigments found in plants of the order Caryophyllales. For example, a plant can be made to express a vividly red betalain via introduction of the reporter gene RUBY (SEQ ID NO: 1), which encodes all the enzymes required for betalain biosynthesis. Thus, in some embodiments, the marker gene is RUBY (SEQ ID NO: 1) or a gene encoding tdTomato.

The marker gene can be located anywhere on the cannabis Y chromosome so long as it is operably linked to a promoter. A “promoter” is a DNA sequence that defines where transcription of a gene begins. RNA polymerase and the necessary transcription factors bind to the promoter to initiate transcription. Promoters are typically located directly upstream (i.e., at the 5′ end) of the transcription initiation site of a gene. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. The term “heterologous promoter” is defined as a promoter that is heterologous to the marker gene. A promoter is “operably linked” to a gene if the promoter is connected to the gene such that it can affect transcription of the gene.

It is understood by those skilled in the art that different promoters direct the expression of a gene in different tissues or cell types, at different stages of development, or in response to different environmental conditions. To be of use in the sorting methods described below, the promoter used with the present invention must drive expression of the marker gene in cannabis seeds and/or pollen. Thus, the promoter may be a constitutive promoter or a tissue-specific promoter that drives expression in cannabis seeds and/or pollen. Suitable promoters for expression in plants include, without limitation, the 35S and 2×35S promoter of the cauliflower mosaic virus, the ubiquitin promoter, the tCUP cryptic constitutive promoter, the Rsyn7 promoter, the maize In2-2 promoter, the tobacco PR-1a promoter, the 7S seed-specific soybean promoter, the phas promoter, the SBP promoter, the USB promoter, the 2S promoter, the 11S promoter, the leB4 promoter, the LEC1a promoter, the actin promoter, the soybean SSU1a promoter, cannabis seed promoters (e.g., Ede1, Ede1p, Ede3), and the lat52 promoter.

### Methods of Sorting Cannabis Seeds or Pollen:

In a second aspect, the present invention provides methods of sorting cannabis seeds or pollen. These methods allow one to control the sex of the plants or the relative proportions of male and female plants produced from the sorted seeds or pollen.

In a first embodiment, the methods are for sorting cannabis seeds. These methods comprise: (a) obtaining a plurality of cannabis seeds comprising seeds that comprise a Y chromosome comprising a marker gene; and (b) sorting the seeds to separate the seeds that comprise the Y chromosome comprising the marker gene from seeds that do not comprise the Y chromosome comprising the marker gene. In these methods, the marker gene encodes a marker protein that must be expressed in seeds to allow for seed sorting. Thus, the marker gene is operably linked to a promoter that drives expression of the marker protein. The seeds that express the marker protein (i.e., the seeds that comprise the Y chromosome comprising the marker gene) can be separated from the seeds that do not express the marker protein (i.e., the seeds that do not comprise the Y chromosome comprising the marker gene) as a means of sorting male seeds from female seeds. These methods may allow for male and female seeds to be separated with 70%, 75%, 80%, 85%, 90%, 95% or even 100% accuracy.

In a second embodiment, the methods are for sorting cannabis pollen. These methods comprise: (a) obtaining a plurality of cannabis pollen grains comprising pollen grains that comprise a Y chromosome comprising a marker gene; and (b) sorting the pollen grains to separate the pollen grains that comprise the Y chromosome comprising the marker gene from the pollen grains that do not comprise the Y chromosome comprising the marker gene. In these methods, the marker gene encodes a marker protein that must be expressed in pollen to allow for pollen sorting. Thus, the marker gene is operably linked to a promoter that drives expression of the marker protein in pollen. The pollen grains that express the marker protein (i.e., the pollen grains that comprise the Y chromosome comprising the marker gene) can be separated from the pollen grains that do not express the marker protein (i.e., the pollen grains that do not comprise the Y chromosome comprising the marker gene) as a means of sorting male pollen grains from female pollen grains. These methods may allow for male and female pollen grains to be separated with 70%, 75%, 80%, 85%, 90%, 95% or even 100% accuracy.

Any known method of sorting may be employed. In some embodiments, the cannabis seeds and/or pollen are sorted using a sorting machine. Examples of suitable seed sorting machines include the TOMRA 3C, which can detect subtle differences in color and shape, optimal sorters such as the SORTEX from the Baler Group, fluorescent sorters such as the COPAS Plus from Union Biometrica, and hyperspectral sorters such as the SeQso iXeed. Example of suitable pollen sorting machines include the Cleanopollen device. In other embodiments, marker gene expression is evaluated using an imaging device, and the seeds or pollen are sorted by hand. The imaging device may use an LED light to activate fluorescent markers that can then be seen with a wide pass filter and used to sort the seeds/pollen. One example of such an imaging device is the NIGHTSEA™ Dual Fluorescent Protein Flashlight.

Only female cannabis plants produce buds and flowers, which are the most abundant source of the lucrative cannabinoids tetrahydrocannabinol (THC) and cannabidiol (CBD), among many others. THC is the main psychoactive compound found in marijuana; it produces the “high” sensation. CBD has been touted for its ability to treat a wide variety of health issues. Thus, in some embodiments, the methods further comprise selecting only female seeds/pollen grains (i.e., seeds/pollen grains that do not comprise the Y chromosome).

To produce cannabis seed (e.g., for propagation), male plants or masculinized female plants are required to provide pollen to fertilize the female plants. Thus, for some applications, it is advantageous to maintain a small number of male plants in the plant population. Therefore, in other embodiments, the methods further comprise selecting a mixture of (a) male seeds and/or pollen grains (i.e., seeds and/or pollen grains that comprise the Y chromosome) and (b) female seeds and/or pollen grains (i.e., seeds and/or pollen grains that do not comprise the Y chromosome). In these embodiments, the female seeds and/or pollen grains may make up at least 75%, at least 80%, at least 85%, at least 90%, at least 95%, or at least 99% of the mixture.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

### Example 1

The following proof of concept example describes the transformation of Cannabis with Plasmid RC6322A (SEQ ID NO: 2) comprising the 2×35S promoter of the cauliflower mosaic virus driving the RUBY marker cassette (SEQ ID NO: 1) gene expression, wherein the cassette is inserted onto the X chromosome.

### Results:

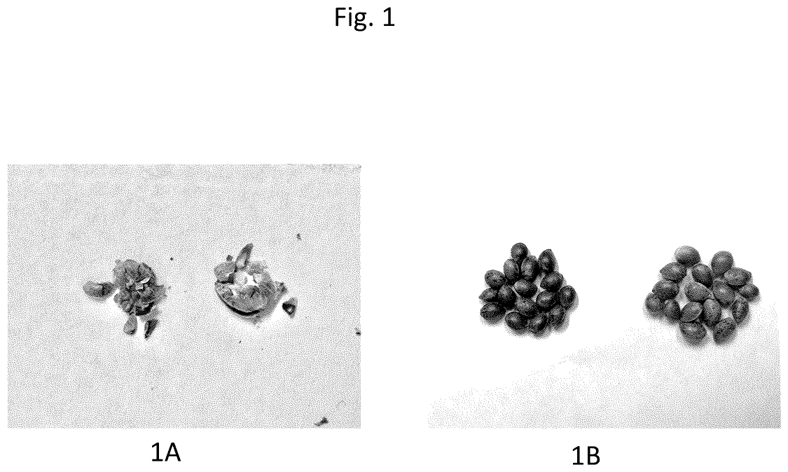

RUBY (SEQ ID NO: 1) expression is visible in mature seeds (FIG. 1), appearing red in color in crushed seeds (FIG. 1A) and visible through the seed coat of intact seeds (FIG. 1B), causing the seed coat to appear darker in color, as compared to control seeds that are negative for expression of the RUBY (SEQ ID NO: 1) marker.

An additional experiment could comprise transformation of a Cherry Wine line and/or different cannabis lines with a control plasmid comprising the 2×35S promoter of the cauliflower mosaic virus driving the RUBY (SEQ ID NO: 1) marker cassette gene expression, wherein the cassette is inserted onto the Y chromosome.

### Materials and Methods:

Plant Material: Badger (female) hemp seeds were imbibed overnight and embryos containing meristematic tissues were isolated (per U.S. Patent Publication No. 2021/0254083).

Construct Development: A plasmid, RC6322A (SEQ ID NO: 2), was constructed comprising the 2×35s promoter driving a RUBY gene cassette (SEQ ID NO:1).

### References

1. Peterson, M. W., Williams, E. J., Harnish, R., Kaeppler, H. F., Martinell, R. C., McFarland, F., & Kaeppler, S. M. Methods of gene editing and transforming cannabis (U. S. patent Ser. No. 11/512,320). U.S. Patent and Trademark Office. Issued Nov. 29, 2022.

### Example 2

An additional experiment could comprise transforming cannabis with a plasmid comprising the CsEde1p seed promoter driving the RUBY marker cassette (SEQ ID NO: 1) gene expression, wherein the plasmid may or may not comprise Ac-Ds transposable elements, which allow for transposition of the cassette to a location other than the original insertion site. A plasmid without an associated transposable element generally is fixed in its position once integrated into the plant genome. Cannabis has 10 pairs of chromosomes and only 1 of those pairs is XY, so the frequency of inserting a piece of DNA into the Y chromosome is low. Including a transposable element will allow the inserted DNA to move from its original location to potentially into the Y chromosome.

### Materials and Methods:

Badger (female) hemp seeds may be imbibed overnight and embryos containing meristematic tissues were isolated (per U.S. Patent Publication No. 2021/0254083).

Construct Development: A plasmid is being constructed with the CsEde1p seed promoter driving a RUBY gene cassette (SEQ ID NO: 1). The plasmid also comprises Ac-Ds transposable elements.

### References

1. Grevelding, C., Becker, D., Kunze, R., Von Menges, Fantes, V., Schell, J., & Masterson, R. High rates of Ac/Ds germinal transposition in Arabidopsis suitable for gene isolation by insertional mutagenesis. Proc. Natl. Acad. Sci. USA. 89, 6085-6089 (1992).
2. Ipek, A., Masson, P. & Simon, P. W. Genetic Transformation of an Ac/Ds-based Transposon Tagging System in Carrot (Daucus carota L.). Europ. J. Hort. Sci., 71 (6). S. 245-251 (2006).
3. Li, Y., Segal, G., Wang, Q., & Dooner, H. K. 2013. Gene Tagging with Engineered Ds Elements in Maize. Methods Mol Biol. 1057, 83-89 (2013).
4. Peterson, M. W., Williams, E. J., Harnish, R., Kaeppler, H. F., Martinell, R. C., McFarland, F., & Kaeppler, S. M. Methods of gene editing and transforming cannabis (U.S. patent Ser. No. 11/512,320). U.S. Patent and Trademark Office. Issued Nov. 29, 2022.

### Example 3

An additional experiment could comprise transforming hemp pollen with a RUBY gene cassette (SEQ ID NO: 1).

### Example 4

An additional experiment could comprise transforming hemp with a gene editing construct (CRISPR/CAS system; CRISPR/Combo system; Talons; Prime editing; Base editing; Zinc Finger Nucleases (ZFNs); Homing endonucleases or meganucleases, where the editing of an endogenous gene, promoter, or other expression element cause the endogenous gene to show a detectable phenotype. Examples could include knocking out genes such as WRI1, KNAT7, NAC2, TTG2, STK, C2H2-like, bZIP44, SHP1, GBF6, PPO, CHS, P, Z, or Bip. Other examples would be to enhance WRI1, KNAT7, NAC2, TTG2, STK, C2H2-like, bZIP414, SHP1, GBF6, PPO, CHS, P, Z, or Bip genes by altering their promoter element or other expression element through and editing system like CRiSPR Combo or others, to likewise cause this gene to show a detectable phenotype in the plant, seed, or pollen grain.

### References

1. Pan, C., Li, G., Malzahn, A. A., Cheng, Y., Leyson, B., Sretenovic, S., Gurel, F., Coleman, G. D., & Qi, Y. Boosting plant genome editing with a versatile CRISPR-Combo system. Nat Plants. 2022 May; 8(5), 513-525.

What is claimed:

1. A cannabis plant, seed, or pollen grain comprising a Y chromosome, wherein the Y chromosome comprises a heterologous promoter operably linked to a marker gene and capable of expressing a marker or wherein a gene is altered through gene editing to generate a marker gene and the marker gene imparts a detectable phenotype in the plant, seed, or pollen grain.

2. The cannabis plant, seed, or pollen grain of claim 1, wherein the marker gene is expressed in seeds or pollen grains.

3. The cannabis plant, seed, or pollen grain of claim 1, wherein expression of the marker gene produces a morphological difference or a detectable signal.

4. The cannabis plant, seed, or pollen grain of claim 3, wherein the marker gene is RUBY (SEQ ID NO: 1) or a gene encoding tdTomato.

5. The cannabis plant, seed, or pollen grain of claim 1, wherein the marker gene is selected from RFP, GFP, YFP, CFP, meffRed, eforRed, aasPink, spisPink, scOrange, crtB, crtI, crtE, CYP76AD1, DODA, or GT.

6. The cannabis plant, seed, or pollen grain of claim 1, wherein the marker gene is selected from WRI1, KNAT7, NAC2, TTG2, STK, C2H2-like, bZIP44, SHP1, GBF6, PPO, CHS, P, Z, or Bip, and optionally wherein the corresponding native marker gene function is knocked out or knocked down to allow detection of the marker.

7. The cannabis plant, seed, or pollen grain of claim 1, wherein the promoter is the ubiquitin promoter, 35S promoter, 2×35S promoter, 7S seed-specific soybean promoter, Ede1 seed promoter, or the CsEde1p seed promoter.

8. A method of sorting cannabis seeds, the method comprising:
a) obtaining a plurality of cannabis seeds comprising the seed of any one of the preceding claims; and
b) sorting the seeds to separate the seeds that comprise the Y chromosome comprising the marker gene from the seeds that do not comprise the Y chromosome comprising the marker gene;
wherein the marker is expressed in seeds to allow sorting.

9. The method of claim 8, further comprising selecting seeds that do not comprise the Y chromosome.

10. The method of claim 8, further comprising selecting a mixture of (a) seeds that comprise the Y chromosome and (b) seeds that do not comprise the Y chromosome.

11. The method of claim 10, wherein the seeds that do not comprise the Y chromosome make up at least 75% of the mixture.

12. A method of sorting cannabis pollen, the method comprising:
a) obtaining a plurality of cannabis pollen grains comprising the pollen grain of any one of claims 1-7; and
b) sorting the pollen grains to separate the pollen grains that comprise the Y chromosome comprising the marker gene from the pollen grains that do not comprise the Y chromosome comprising the marker gene;
wherein the marker is expressed in pollen grains to allow sorting.

13. The method of claim 12, further comprising selecting pollen grains that do not comprise the Y chromosome.

14. The method of claim 12, further comprising selecting a mixture of pollen grains that comprise the Y chromosome and pollen grains that do not comprise the Y chromosome.

15. The method of claim 14, wherein the pollen grains that do not comprise the Y chromosome make up at least 75% of the mixture.

16. The method of claim 12, wherein the marker gene is selected from RFP, GFP, YFP, CFP, meffRed, eforRed, aasPink, spisPink, scOrange, crtB, crtI, crtE, CYP76AD1, DODA, or GT.

17. The method of claim 12, wherein the marker gene is selected from WRI1, KNAT7, NAC2, TTG2, STK, C2H2-like, bZIP44, SHP1, GBF6, PPO, CHS, P, Z, or Bip, and optionally wherein the native gene function is knocked out or knocked down to allow detection of the marker.

18. The method of claim 8, wherein expression of the marker gene produces a morphological difference or a detectable signal.

19. The method of claim 18, wherein the marker gene is RUBY (SEQ ID NO: 1) or a gene encoding tdTomato.

20. The method of claim 8, wherein the marker gene is RFP, GFP, YFP, CFP, meffRed, eforRed, aasPink, spisPink, scOrange, crtB, crtI, crtE, CYP76AD1, DODA, or GT.

21. The method of claim 8, wherein the marker gene is selected from WRI1, KNAT7, NAC2, TTG2, STK, C2H2-like, bZIP44, SHP1, GBF6, PPO, CHS, P, Z, or Bip, and optionally wherein the native gene function is knocked out if necessary to allow detection of the marker.

22. The method of claim 8, wherein the promoter is the ubiquitin promoter, 35S promoter, 2×35S promoter, 7S seed-specific soybean promoter, Ede1 seed promoter, or the CsEde1p seed promoter.


SEQUENCE LISTING (2 sequences)

SEQ ID NO: 1 - DNA, 3975 bp, synthetic construct
  source 1..3975: mol_type = other DNA
atggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaagtcctcggcaaggacaagcagatccaagagtccgacatcatcaacctcccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggttcgggttctggacagctgttgaattttgaccttcttaagcttgcgggtgacgtcgagtccaaccctgggccaggaatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcgggttctggagagggcaggggatctcttctaacatgcggggacgtggaggaaaatcccggcccaggaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccggaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagggttcgtaa

SEQ ID NO: 2 - DNA, 14993 bp, synthetic construct
  source 1..14993: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggtatcgataattgtaaatgtaattgtaatgttgtttgttgtttgttgttgttggtaattgttgtaaaaatgagctcttatacagtagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaaggagaagttgtaatgagttgctggcctctcttgggattgttcatcaaagtagctgtccctacacaccacaacagaatggtgttgtggagagaaagcacagacacatccttgagatggcaagggcacttaagtttcagagtggtgtacctaccaggttttggggagactgtgtcaagactgttgtatacctgatcaatagactccctactccaatcttacaaggcaaatgtccatatgaactactctatcagaaacatgccaagcttgatcacttgagagtgtttgggtgtcttgcttttgcagcaacactgcccaaaggtgacaaacttgcacctagagccaagcgaaccatcttcattggttattctgagactcaaaagggttacaggttgtatgacttggataataaggtgatcatagtgagcagggatgtagttttcagagagtttcagttccctttcaaagagtccatctcccatgaacctgatatgttcactcgagcaggttcacttagctctgaagatacagctttgcagctagtgtataatgatatttttccttccagctttcatactgatgatgatgatactccagttcttattcctgacattgtagaggccaatgaggatatcactttggggactcattctacccaaaatcatatcacttcagctgatgctgcaccttcaggccacctgcatgctcctgcagagtcagttgagtcagctgagcctgacctagcaaatgctgaatttgagcaccatactgctgtagctgatccctctctagttccagcaaacctgcacactagacctaaacgcaatgcaggtcctcccatctggctcaaggactttgtgacactgaacaaaggctctagggatgttccataccctatagccaactatgtttcctatgatcacttgcctgttcactatcaagcttatttgagtgctttctccacttatactgaacctaaatccttcaaagaagcagctcaagatgagaaatggatggaggccatgtcccttgagatacaggctcttgaggataataacacctgggagattgtccctttaccccctggtaaacagcctatagggtccaaatgggtgtacaaaattaaatacaaagctaatggtgaagttgacaggtttaaggcaaggctagtggccaagggatacactcagcaagaaggccttgactaccatgaaactttttctccagtggccaaaatggtcactgtaaaagctgtcatatctgttgctgcttccaagggctggttccttttccagatggatgtcaacaatgcttttctacaaggtgacctcatggaagaagtttatatgtctctgcctcagggttttcacagccaaggggaaaccaaagtgtgcaggctgttgaatccctctatggtttaaagcaagcatcaaggcagtggaacatcaagcttaccactgtccttatgcaggctggttttatgcaaagtgcttatgatcactccttgtttaccaaaagagaggggactgaccttgtcataatcctgatttatgtggatgatctgttaataacaggcagcagcaatgttttgatttcagaagcaaaggcaaccctgcatcagcatttcaagatgaaagatttgggagaactaaaatacttcttaggcattgaggtgctgagatcagaaaagggaatcctactgaaccagaggaagtatgcactggaactgatatcaggtgttggtctgggaggctgcagaccagtgtcaaccccaatggaacagaaccaaaggctgactactgtagaatatgacaagcacttaggaaagactgatgatgcagagttagaggatgtgggatcctatcagagactggtgggaaagcttctctacttgacaatcacaaggccagatatcagctttgcagtgcaggtgctgagtcaattcatgcagcaacccaaacagtcacacctggaagcagcattgagggttgtgaagtacataaaaggttctccaggtgttaggaacgctactagaattcgagctcggagttacgtgtgctgtgagacattatcatcaattgtgttgtatatgatatatagatatagatatataaatatatagattgagtgatataatatatttaaaatataaattatatatatgttttaatatatttttgcatatatatatatatttgtaaaaactagaagtattttttcatgagataattattatcgagttgaataagtctattatttgtgagagccaaccatatttatatatgtgattaaattttatctttgtgaaattaaaaataataaaaaataccttaaaatcataataatagaaaaacttatatttataatttaccattatacttaaaaaaaattaaataaatattataaatataaatactatcgagtaatggccgcgctagggtttttgagaaaaaatcttcccacgcactcaactgcactgtacggcgtcgttttcacagccgcataatagaagccgcgttccccaacccttcctcacaacattctcggaccctccagcaccgtcacccaaacaaatatccacgcggtagtaggcgcgtgaaacaaactctaatccgaactacgagtcgtgagaagcacgcgctttagcgagcgtttcaattgtcgctacgaaagcagagaaggatacaaacggaactagggtaaattagtaagggtaatttcgtaaacagaagaaaagagttgtagctataaataaaccctctaaccctcgtcgcattacttctcttcacacctttgttcactcttcttctcttgcggctagggttttagcgcagcttcttctaggttcgttatctaccaccgttctatggattttattccttctattcgtgtttattctattggtttatgttgcttgcaatatgttttttctgaatctgtcgtcgttgacttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcagatttaaggaagaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaagtcctcggcaaggacaagcagatccaagagtccgacatcatcaacctcccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggttcgggttctggacagctgttgaattttgaccttcttaagcttgcgggtgacgtcgagtccaaccctgggccaggaatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcgggttctggagagggcaggggatctcttctaacatgcggggacgtggaggaaaatcccggcccaggaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccggaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagggttcgtaagcttatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggactcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatcgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct